SEQUENCE LISTING (3 sequences)

SEQ ID NO: 1 - protein, 748 aa, Homo sapiens
MSNPGGRRNGPVKLRLTVLCAKNLVKKDFFRLPDPFAKVVVDGSGQCHSTDTVKNTLDPKWNQHYDLYIGKSDSVTISVWNHKKIHKKQGAGFLGCVRLLSNAINRLKDTGYQRLDLCKLGPNDNDTVRGQIVVSLQSRDRIGTGGQVVDCSRLFDNDLPDGWEERRTASGRIQYLNHITRTTQWERPTRPASEYSSPGRPLSCFVDENTPISGTNGATCGQSSDPRLAERRVRSQRHRNYMSRTHLHTPPDLPEGYEQRTTQQGQVYFLHTQTGVSTWHDPRVPRDLSNINCEELGPLPPGWEIRNTATGRVYFVDHNNRTTQFTDPRLSANLHLVLNRQNQLKDQQQQQVVSLCPDDTECLTVPRYKRDLVQKLKILRQELSQQQPQAGHCRIEVSREEIFEESYRQVMKMRPKDLWKRLMIKFRGEEGLDYGGVAREWLYLLSHEMLNPYYGLFQYSRDDIYTLQINPDSAVNPEHLSYFHFVGRIMGMAVFHGHYIDGGFTLPFYKQLLGKSITLDDMELVDPDLHNSLVWILENDITGVLDHTFCVEHNAYGEIIQHELKPNGKSIPVNEENKKEYVRLYVNWRFLRGIEAQFLALQKGFNEVIPQHLLKTFDEKELELIICGLGKIDVNDWKVNTRLKHCTPDSNIVKWFWKAVEFFDEERRARLLQFVTGSSRVPLQGFKALQGAAGPRLFTIHQIDACTNNLPKAHTCFNRIDIPPYESYEKLYEKLLTAIEETCGFAVE

SEQ ID NO: 2 - DNA, 37 bp, Artificial Sequence
  forward primer used for PCR cloning of mTRAF2
cgggatccgcggccgctatggctgcagccagtgtgac

SEQ ID NO: 3 - DNA, 47 bp, Artificial Sequence
  reverse primer used for PCR cloning of mTRAF2
gcatagtttagcggccgcttatctagagagtcctgttaggtccacaa

(19) United States
(12) Patent Application Publication
(10) Pub. No.: US 2006/0134737 A1
(43) Pub. Date: Jun. 22, 2006

(54) Ubiquitinated TNF receptor2 and its uses

(75) Inventors: Rudi Beyaert, Zingem (BE); Isabelle Carpentier, Gent (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Universiteit Gent, Gent (BE)

(21) Appl. No.: 11/320,424

(22) Filed: Dec. 28, 2005

Related U.S. Application Data
(63) Continuation of application No. PCT/EP04/51327, filed on Jul. 1, 2004.

(30) Foreign Application Priority Data
Jul. 1, 2003 (EP) 03101956.5

(51) Int. Cl. C07K 14/715; C07H 21/04; C12P 21/06

(52) U.S. Cl. 435/68.1; 435/69.1; 435/320.1; 435/325; 530/350; 536/23.5

(57) ABSTRACT

The present invention relates to ubiquitinated TNF receptor 2, and the uses thereof. More specifically, the invention relates to the use of TNF receptor 2 ubiquitination to deplete TNF receptor 2 from the cell membrane and cytoplasm, and relocalize it in the insoluble cell fraction. Such relocalization can be used to modulate the signaling activity of the TNF receptor 2 and to treat TNF receptor 2-related diseases. The invention relates further to the use of Smurf 2 to ubiquitinate TNF receptor 2 and to the use of TRAF2 to mediate TNF receptor 2 ubiquitination.

### CROSS-REFERENCE TO RELATED APPLICATIONS

This application is a continuation of PCT International Patent Application No. PCT/EP2004/051327, filed on Jul. 1, 2004, designating the United States of America, and published, in English, as PCT International Publication No. WO 2005/003167 A1 on Jan. 13, 2005, which application claims priority to U.S. Provisional Patent Application Ser. No. 60/484,232 filed Jul. 1, 2003, and to European Patent Application Serial No. 03101956.5, filed Jul. 1, 2003, the contents of each of which are hereby incorporated by this reference. This patent application also claims the benefit under 35 U.S.C. § 119(3) of U.S. Provisional Patent Application Ser. No. 60/484,232 filed Jul. 1, 2003.

### TECHNICAL FIELD

The present invention relates generally to biotechnology such as an ubiquitinated TNF receptor 2 (TNF-R2), and the uses thereof. More specifically, the invention relates to the use of TNF-R2 ubiquitination to deplete TNF-R2 from the cell membrane and cytoplasm, and relocalize it in the insoluble cell fraction. Such relocalization can be used to modulate the signaling activity of the TNF-R2 and to treat TNF-R2-related diseases. The invention relates further to the use of Smurf 2 to ubiquitinate TNF-R2 and to the use of TRAF2 to mediate TNF-R2 ubiquitination.

### BACKGROUND

TNF is the prototypic member of the TNF ligand family, with a key role in several processes, such as inflammation, cell proliferation and cell killing (Kollias et al., 1999; Bodmer et al., 2002). TNF exerts these functions by binding to two distinct receptors known as TNF-R1 and TNF-R2. Several proteins that bind directly or indirectly with the cytoplasmic tails of TNF-R1 and TNF-R2 have been described, connecting these receptors to signaling cascades leading to apoptosis, activation of the transcription factor nuclear factor-κB (NF-κB), and the activation of c-jun N-terminal kinase (JNK) and p38 mitogen-activated protein kinase (p38 MAPK) (reviewed by Kyriakis and Avruch, 2001; Carpentier et al., 2003). Of special interest are TRAF 1 and 2, which were originally identified by affinity chromatography as TNF-R2-associated proteins (Rothe et al., 1994). They are the founders of a new protein family known as the TRAF-family, currently expanded to six family members (TRAF1-6) in mammalians. They are characterized by an N-terminal “really interesting novel gene” (RING) domain, followed by several zinc fingers (except for TRAF1), and a C-terminal TRAF-domain that can be subdivided into a coiled-coil TRAF-N domain and a more conserved TRAF-C domain (for review, see Chung et al., 2002). TRAF1 and TRAF2 can form a homo- or heteromeric complex with the TRAF-binding domain of TNF-R2 (Rothe et al., 1994). It is generally accepted that TRAF2 mediates TNF-R2-induced p38 MAPK/JNK, as well as NF-κB activation (Rothe et al., 1995; Reinhard et al., 1997; Jupp et al., 2001), whereas TRAF1 might fulfill an inhibitory role (Carpentier and Beyaert, 1999; Tsitsikov et al., 2001). Overexpression experiments indicated a requirement for the RING finger of TRAF2 for its function (Rothe et al., 1995; Reinhard et al., 1997). In contrast to TNF-R2-induced NF-κB and p38 MAPK/JNK activation, the mechanism of TNF-R2-induced apoptosis is much less clear. Although TNF-R2 can directly initiate pro-apoptotic signaling (Zheng et al., 1995), TNF-R2 can also play an important regulatory role because TNF-R2 can enhance TNF-R1-induced apoptosis by a TRAF2-dependent mechanism (Duckett and Thompson, 1997; Weiss et al., 1997 and 1998; Declercq et al., 1998; Chan and Lenardo, 2000). TNF-R2 stimulation leads to the cytoplasmic depletion of TRAF2, thereby disabling the recruitment of anti-apoptotic proteins to TNF-R1 (Fotin-Mleczek et al., 2002).

Ubiquitination is a post-translational modification that involves the covalent attachment of one or more ubiquitin moieties to target proteins. Poly-ubiquitination mainly targets the proteins to the proteasomal degradation pathway, whereas mono-ubiquitination regulates other processes, such as endocytosis of membrane proteins (reviewed in Glickman and Ciechanover, 2002 and Hicke, 2001a and b). These processes are achieved by the concerted action of three enzymes, known as the E1 ubiquitin-activating enzyme, the E2 ubiquitin-conjugating enzymes and the E3 ubiquitin ligases. The latter ones are mainly responsible for the substrate specificity. Three types of ubiquitin ligases have been defined based on their catalytic domains: the RING finger, the U-box and the HECT domain E3s (reviewed by Pickart, 2001). Smurf 1 and 2 were recently identified as novel members of the HECT family. Besides a C-terminally located catalytic domain, their structure comprises an N-terminal C2 domain followed by 2 (Smurf1) or 3 (Smurf2) tryptophan-rich (WW) domains. Both Smurfs have been implicated in the negative regulation of TGF-β signal transduction in two different ways. First, they act on receptor-Smads (R-Smads): both Smurfs induce the ubiquitination and subsequent degradation of the R-Smad Smad1 in a ligand-independent fashion, whereas the action of Smurf2 on R-Smad Smad2 seems to be restricted to the TGF-β-activated pool of Smad2 (Zhu et al., 1999; Lin et al., 2000; Zhang et al., 2001; Bonni et al., 2001). Secondly, both Smurfs use the inhibitory Smad (1-Smad) Smad7 to become targeted to the TGF-βR complex, where they are responsible for the concomitant lysosomal and proteasomal turnover of both adaptor and receptor (Kavsak et al., 2000; Ebisawa et al., 2001). In contrast, Smurf2 has also been reported to intensify TGF-β signaling by degrading the transcriptional co-repressor SnoN (Bonni et al., 2001).

### SUMMARY OF THE INVENTION

Surprisingly, we have found that the action of Smurf2 is not restricted to TGF-β signal transduction, but extends to the TNF-R2 pathway. Smurf2 was isolated as a novel interaction partner of TRAF2, and induces the ubiquitination and redistribution of TNF-R2. In contrast, a ligase inactive Smurf2 point mutant had no effect on TNF-R2. Moreover, TNF treatment also resulted in ubiquitination and relocalization of TNF-R2. These results indicate that TRAF2 acts as an adaptor protein that recruits Smurf2 to TNF-R2, leading to its ubiquitination and relocalization.

A first aspect of the invention is a TNF-R2 comprising SEQ ID NO:1, or a functional fragment thereof, wherein TNF-R2 or functional fragment is ubiquitinated. “Ubiquitinated” as used herein indicates that at least one ubiquitin molecule is bound to the receptor or functional fragment; however, it includes mono-ubiquitination, as well as poly-ubiquitination, wherein several ubiquitin molecules are linked at one site, or one or more molecules are linked at multiple sites.

Another aspect of the invention is the use of Smurf2 to ubiquitinate TNF-R2. Smurf2 is a member of the E3 ubiquitin-protein-ligase protein family, comprising a HECT catalytic domain. It is known that the E3 ligases like Smurf2 confer the specificity to the protein ubiquitination; however, on the base of the sequence, it cannot be forecasted which proteins will be ubiquitinated by Smurf2, and TNF-R2 ubiquitination by Smurf2 is unexpected.

Still another aspect of the invention is the use of TRAF2 to mediate TNF-R2 ubiquitination. “Mediation,” as used herein, means that TRAF2 itself is not ubiquitinating the TNF-R2, but does play a role in the ubiquitination of the TNF-R2, e.g., as “docking” protein, to facilitate the binding of Smurf2 with TNF-R2.

Another aspect of the invention is the use of ubiquitination of TNF-R2 to relocalize TNF-R2 to the insoluble cell fraction. Still another aspect of the invention is the use of Smurf2 and/or TRAF2 to relocalize TNFR2 to the insoluble cell fraction. Indeed, it is shown in this invention that Smurf2 is ubiquitinating TNF-R2, mediated by TRAF2. As this ubiquitinated TNF-R2 is relocalized into the insoluble cell fraction, Smurf2 and/or TRAF2 can be used to relocalize TNF-R2.

As relocalization of the TNF-R2 to the insoluble cell fraction will influence the signaling activity, another aspect of the invention is the use of TNF-R2 ubiquitination to modulate TNF-R2 signaling. Still another aspect of the invention is the use of TNF-R2 ubiquitination to modulate apoptosis. Indeed, although TNF-R2 does not contain a DD that is present in TNF-R1 and many other apoptosis-inducing receptors, TNF-R2 does play an important role in TNF-induced apoptosis. The use of agonistic TNF-R2-specific antibodies or TNF-R2-specific muteins clearly established that exclusive triggering of this receptor is sufficient to induce cell death in some cell lines (Heller et al., 1992; Grell et al., 1993; Bigda et al., 1994; Medvedev et al., 1994; Haridas et al., 1998), or to cooperate with TNF-R1 (Vandenabeele et al., 1995, Leeuwenberg et al., 1995). For instance, a role of TNF-R2 in TCR-induced cell death of activated CD8⁺ T cells was deduced from experiments using TNF-R2-deficient mice (Zheng et al., 1995). Likewise, in AIDS patients, the decreased number of CD8⁺ T cells is due to TNF-R2-mediated apoptosis (Herbein et al., 1998). Still another aspect of the invention is the use of TNF-R2 ubiquitination to modulate proliferation of peripheral blood mononuclear cells (Gehr et al., 1992), natural killer cells (Mason et al., 1995), B cells (Erikstein et al., 1991), oligodendrocyte precursors (Arnett et al., 2001), thymocytes (Tartaglia et al., 1991) and peripheral T cells (Tartaglia et al., 1993, Kim and Teh, 2001). TNF-induced proliferation of these cells is exclusively mediated by TNF-R2. TNF-R2 seems to provide an important costimulatory signal, in addition to CD28, towards optimal T cell proliferation (Kim and The, 2001).

### BRIEF DESCRIPTION OF THE FIGURES

FIG. 1. Panel A is a schematic representation of Smurf2. Abbreviations used: C2, Ca²⁺/phospholipid binding domain; WW, tryptophan-rich domain; HECT, homologous to E6-AP carboxyl terminus domain; C* represents the catalytic cysteine residue. Panel B shows TRAF2 co-immunoprecipitates with Smurf2. Hek293T cells were transiently transfected with the expression plasmids encoding for HA-tagged Smurf2 and E-tagged TRAF2. Cell lysis was performed in Smurf2 lysis buffer (10 mM Hepes pH 7.9, 300 mM NaCl, 0.1 mM EGTA, 20% glycerol, 0.2% NP-40) supplemented with protease- and phosphatase-inhibitors. Lysates were incubated with anti-HA antibody and immobilized on protein A trisacryl beads. Beads were washed six times with RIPA buffer. Co-precipitated proteins were analyzed by Western blotting with anti-E-tag antibody (upper panel). Expression levels of HA-Smurf2 and E-TRAF2 were controlled with anti-HA (middle panel) and anti-E-HRP antibody, respectively (lower panel). * represents a non-specific band. Panel C depicts co-immunoprecipitation of TRAF2 with deletion mutants of Smurf2. Hek293T cells were transiently transfected with E-TRAF2 and the expression plasmids encoding for Flag-tagged Smurf2 or deletion mutants. Cell lysis was performed in TNF-R2 lysis buffer supplemented with protease- and phosphatase-inhibitors. Lysates were incubated with anti-Flag antibody and immobilized on protein A trisacryl beads. Beads were washed twice with lysis buffer, twice with lysis buffer containing 0.75 M NaCl and twice with lysis buffer. Immunoprecipitation was performed with anti-Flag antibody followed by Western blot detection with anti-E-tag antibody (upper panel). Expression levels of E-TRAF2 and Flag-Smurf2 or deletion mutants thereof were controlled with anti-E-HRP (middle panel) and anti-Flag-HRP antibody (lower panel), respectively. Abbreviations used: IP, immunoprecipitation.

FIG. 2. Smurf2 does not affect TRAF2 expression. 2×10⁵ HEK293T cells were transiently transfected with increasing amounts (0, 50, 100, 200, 500, 1000 ng) of an expression plasmid encoding HA-Smurf2. Thirty-six to 48 hours later, cell lysis was performed in 200 μl RIPA buffer, followed by SDS-PAGE and immunoblotting with anti-TRAF2 (upper panel) and anti-HA (lower panel) antibody.

FIG. 3. Smurf2 induces a decrease in TNF-R2 levels in the soluble cell fraction. 2×10⁵ HEK293T cells were transiently transfected with 100 ng expression plasmids encoding for TNF-R2 and GFP, and increasing amounts (0, 50, 100, 200, 500, 1000 ng) of HA-Smurf2. Thirty-six to 48 hours later, cell lysis was performed in 200 μl RIPA buffer, followed by SDS-PAGE and immunoblotting with anti-TNF-R2, anti-HA, anti-GFP and anti-TRAF2 antibody. The multiple bands for TNF-R2 might represent differently glycosylated forms of TNF-R2.

FIG. 4. Smurf2, but not Smurf2 (C716G), induces a decrease in TNF-R2 levels in the soluble cell fraction. 2×10⁵ HEK293T cells were transiently transfected with 100 ng expression plasmids encoding for TNF-R2 or caspase 14APro (as a negative control), and 1000 ng of HA-Smurf2 WT or cMyc-Smurf2 (C716G). Lysis was performed in 200 μl RIPA buffer, followed by SDS-PAGE and immunoblotting with anti-TNF-R2, anti-caspase 14, anti-HA and anti-cMyc antibody.

FIG. 5. Smurf2 has no effect on TNF-R2 mRNA levels. 2×10⁵ HEK293T cells were transfected with expression plasmids encoding for 100 ng TNF-R2 and/or 1 μg Smurf2 (WT or C716G). The next day, total RNA was isolated, 10 μg was loaded and separated on gel, blotted, and probed with TNF-R2. Expression of GAPDH served as a control for the quantity of RNA loaded.

FIG. 6. TRAF2 functions as an adaptor between TNF-R2 and Smurf2. Thirty-six to 48 hours after transient transfection of HEK293T cells with 1 μg TNF-R2, 1 μg cMyc-Smurf2 (C716G) and increasing amounts of E-TRAF2 (0, 0.25, 1 or 2 μg), cells were lysed in TNF-R2 lysis buffer. TNF-R2 was immunoprecipitated with utr-1 antibody. Co-immunoprecipitated E-TRAF2 and cMyc-Smurf2 (C716G) were revealed by Western blotting with anti-E-HRP and anti-cMyc antibody, respectively (upper two panels). Aliquots of total cell extracts were immunoblotted with anti-cMyc, anti-E-HRP or anti-TNF-R2 antibody (lower three panels) to confirm expression of the transfected genes. Abbreviations used: IP, immunoprecipitation.

FIG. 7. Smurf2 induces ubiquitination of TNF-R2, but not of TRAF2. 1.2×10⁶ HEK293T cells were transiently transfected with expression plasmids for TNF-R2, TRAF2, HA-Smurf2 WT, c-myc-Smurf2(C716G) as indicated. Immunoprecipitation was performed with utr-1 (left panel) or anti-E (right panel) antibody to immunoprecipitate TNF-R2 and E-TRAF2, respectively; followed by immunoblotting with anti-HA antibody (upper panels) to detect TNF-R2- or TRAF2-HA ubiquitin conjugates. Aliquots of total cell extracts were immunoblotted with anti-TNF-R2, anti-E-HRP or anti-cMyc antibody (lower two panels) to confirm expression of the transfected genes.

FIG. 8. Smurf2 induces the relocalization of TNF-R2 to the insoluble fraction. 2×10⁵ HEK293T cells were transiently transfected with indicated expression plasmids. Soluble, insoluble and total cell fractions were prepared as described in Materials and Methods. Equal amounts of each fraction were separated by SDS-PAGE and analyzed by immunoblotting with anti-TNF-R2 (upper panel), anti-TRAF2 (second panel), anti-HA (third panel) or anti-cMyc (lower panel) antibody.

FIG. 9. TNF stimulation induces ubiquitination of TNF-R2. 1×10⁷ PC60hTNF-R55 R75 cells were pretreated with 30 μM MG-132 for 30 minutes, followed by stimulation with 1 μg/ml hTNF for 15 and 60 minutes. Immunoprecipitation of TNF-R2 was performed with utr-4 antibody, followed by immunoblotting with anti-ubiquitin antibody (upper panel). The same blot was reprobed with anti-TNF-R2 antibody to control TNF-R2 immunoprecipitation (lower panel).

FIG. 10. TNF stimulation induces the relocalization of TNF-R2 to an insoluble fraction. 2×10⁶ PC60hTNF-R55R75 cells were pretreated for 30 minutes with 20 μM MG-132 prior to stimulation with 1 μg/ml hTNF for the indicated times. Equal amounts of each cell fraction were separated by SDS-PAGE and analyzed for TNF-R2 expression by immunoblotting.

FIG. 11. Smurf2 WT and Smurf2 C716G both have an inhibitory effect on TNF-R2-induced NF-κB activity. 2×10⁵ HEK293T cells were transiently transfected with 100 ng pUT651, 100 ng pNFconluc, 100 ng TNF-R2 and varying amounts of Smurf2 WT or Smurf2 (C716G) as depicted. The next day, 1/10^{th} of the cells were seeded out in triplicate in 24-well plates. The following day, cell extracts were analyzed for luciferase and β-galactosidase activity and plotted as luc/β-gal, which is representative for NF-κB activity.

FIG. 12. Smurf2 has no effect on p38 MAPK activation. 2×10⁵ HEK293T cells were transiently transfected with 100 ng of expression plasmids for E-TRAF2 or HA-Smurf2 as indicated. Twenty-four hours later, cells were left untreated or treated with 1000 IU/ml hTNF for ten minutes and lysed in 200 μl p38 MAPK lysis buffer. Activated (phosphorylated) and total amounts of p38 MAPK were determined by immunoblotting with a p38 MAPK phospho-specific antibody (upper panel) or a p38 MAPK antibody (lower panel). Ectopic expression of HA-Smurf2 WT and E-TRAF2 were detected via immunoblotting with anti-HA and anti-E-HRP antibody, respectively.

### DETAILED DESCRIPTION OF THE INVENTION

A further aspect of the invention is the use of TNF-R2 ubiquitination to modulate inflammation and/or autoimmune disease. Preferably, inflammation and/or autoimmune disease is selected from the group consisting of Crohn's disease, rheumatoid arthritis, systemic lupus erythematosus, sepsis, chronic hepatitis virus infection, acute pancreatitis, acute respiratory distress syndrome and AIDS. Indeed, TNF-R shedding results in a soluble receptor corresponding to the extracellular region and alters rapidly the number of functionally active TNF-R. These soluble TNF-Rs, originally identified as TNF-binding proteins, have been found in urine, serum, ovarian ascites and synovial and cerebral spinal fluids of patients with various diseases (Nophar et al., 1990, Cope et al., 1992; Grosen et al., 1993). Several inflammatory disorders are associated with increased production of soluble TNF-Rs, in which a correlation between the disease progression and the soluble TNF-R level can be found. For soluble TNF-R2, these include rheumatoid arthritis (Cope et al., 1992), systemic lupus erythematosus (Gabay et al., 1997), sepsis (Schroder et al., 1995); chronic hepatitis virus infection (Marinos et al., 1995); acute pancreatitis (de Beaux et al., 1996); acute respiratory distress syndrome (Lucas et al., 1997) and AIDS (Hober et al., 1996). Moreover, chronic production of human TNF-R2 in TNF-R2 transgenic mice has detrimental effects leading to multi-organ inflammatory syndrome involving mainly the pancreas, liver, kidney and lung (Douni and Kollias, 1998). In addition, T-cell-deficient SCID mice reconstituted with CD4⁺ CD62L⁺ T cells from TNF-R2 transgenic mice develop more severe experimental colitis than when wild-type T cells were used for reconstitution. This observation correlates with the up-regulated expression of TNF-R2 on the CD4⁺ T cells observed in patients with Crohn's disease (Holtmann et al., 2002).

The invention is further described with the aid of the following illustrative Examples.

### EXAMPLES

Materials and Methods to the Examples

Cell Lines and Reagents

Human embryonic kidney cells (HEK293T) were a kind gift of Dr. M. Hall (University of Birmingham, Birmingham, UK) and were grown in DMEM supplemented with 10% fetal bovine serum, 2 mM glutamin, 0.4 mM sodium pyruvate, 106 U/l penicillin and 100 mg/l streptomycin. PC60hTNF-R55 R75 is a rat T-lymphoma and mouse CTL hybrid cell line stably transfected with human TNF-R1 and 2, as described previously (Vandenabeele et al., 1995). Cells were grown in RPMI 1640 supplemented with 100 μM β-mercaptoethanol, 10% fetal bovine serum, glutamin, sodium pyruvate and antibiotics. The proteasome inhibitor MG-132 was purchased from BioMol Research Laboratories, Inc. (Butler Pike, Pa., USA). Recombinant human TNF was produced in E. coli in our laboratory and purified to 99% homogeneity. TNF has a specific biological activity of 8.8×10⁶ IU/mg purified protein, as determined with the international standard code 87/650 (National Institute for Biological Standards and Control, Potters Bar, UK). The TNF-R2-specific monoclonal agonistic utr-1 and non-agonistic utr-4 antibodies were obtained from Dr. W. Lesslauer (Roche Basel, Basel, Switzerland), polyclonal anti-caspase 14 antibody from S. Lippens (DMBR, Ghent University—VIB, Ghent, Belgium), polyclonal anti-TNF-R2 antibody from Dr. W. A. Buurman (University of Maastricht, the Netherlands), and monoclonal anti-cMyc antibody from Dr. N. Mertens (DMBR, Ghent University—VIB, Ghent, Belgium). Monoclonal anti-HA antibody was purchased from CRP (Richmond, Calif., USA), polyclonal anti-TRAF2 (sc-876) and monoclonal anti-ubiquitin (sc-8017) antibodies from Santa Cruz Biotechnology (Santa Cruz, Calif., USA), anti-GFP (green fluorescent protein) antibody (JL-8) from Clontech (Palo Alto, Calif., USA), monoclonal anti-Flag and monoclonal anti-Flag-horseradish peroxidase (HRP)-linked antibodies from Sigma-Aldrich (St. Lois, Mo., USA), monoclonal anti-E tag and monoclonal anti-E-HRP-linked antibodies from Amersham Pharmacia Biotech (Rainham, UK). Anti-mouse and anti-rabbit HRP-linked antibodies were obtained from Amersham Pharmacia Biotech.

Expression Plasmids

HA-Smurf2 and cMyc-Smurf2 (C716G) cDNA were a kind gift from Dr. Y. Zhang (Laboratory of Cellular and Molecular Biology, National Cancer Institute, Bethesda, Md., USA), and cloned in pCAGGS expression vectors. cMyc-Smurf2 WT was obtained by cutting HA-Smurf2 WT and cMyc-Smurf2 (C716G) with ScaI and ligating the resulting cMyc-containing fragment to the Smurf2 WT-containing fragment. pCMV5B-Flag-Smurf2 WT and several Flag-tagged deletion mutants lacking the C2 domain (Smurf2ΔC2), the first, second or third WW domains or combinations (Smurf2ΔWW1, Smurf2ΔWW2, Smurf2ΔWW3, Smurf2ΔWW2/3), the C2 domain and the first WW domain (Smurf2ΔC2/WW1) or the HECT domain (Smurf2ΔHECT) were a kind gift from Dr. J. Wrana (Samuel Lunenfeld Research Institute, Mount Sinai Hospital, Toronto, Canada). pcDNAI-TNF-R2 WT and mutants were obtained from Dr. W. Declercq (DMBR, Ghent University—VIB, Ghent, Belgium) and were described previously (Declercq et al., 1998). pcDNA3-caspase 14Δpro was kindly given by S. Lippens and pEF6-E by B. Depuydt (DMBR, Ghent University—VIB, Ghent, Belgium). pcDNA3-HA-ubiquitin was kindly provided by Dr. K. DiMarco-Burns (University of Lausanne, Lausanne, Switzerland). pEF6-E-murine (m) TRAF2 was obtained after PCR cloning of mTRAF2 (forward primer (SEQ ID NO:2), 5′-cgggatccgcggccgctatggctgcagccagtgtgac-3′ and reverse primer (SEQ ID NO:3), 5′-gcatagtttagcggccgcttatctagagagtcctgttaggtccacaa-3′), cutting the PCR product with NotI, and cloning into the NotI site of pEF6-E.

Transfection, Co-immunoprecipitation and Western Blotting

For immunoprecipitation, 1.2×10⁶ HEK293T cells were plated on 10-cm Petri dishes and transiently transfected with a total amount of 5 μg DNA using the DNA calcium phosphate coprecipitation method (O'Mahoney and Adams, 1994). 1 μg of each expression plasmid was used, except if mentioned otherwise in the figure legends. Thirty-six to 48 hours later, cells were lysed in the indicated lysis buffer supplemented with protease-(10 μg/ml leupeptin, 200 U/ml aprotinin and 1 mM PMSF) and phosphatase-inhibitors (10 mM NaF, 1 mM Na-vanadate and 5.5 mg/ml β-glycerophosphate). Cell lysates were incubated with the indicated antibodies and immobilized to protein A trisacryl beads (Pierce Chemicals, Rockford, Ill., USA). Beads were washed six times with buffers as indicated in figure legends and binding proteins were eluated with 1× Laemmli buffer. Co-precipitating proteins were separated by SDS-PAGE and analyzed by Western blotting using Renaissance-enhanced chemiluminescence system (NEN, Boston, Mass., USA).

Northern Blot Analysis

2×10⁵ HEK293T cells were transiently transfected with 100 ng TNF-R2 and/or 1 μg HA-Smurf2 WT or cMyc-Smurf2 (C716G). The following day, total RNA was isolated via RNAzol (WAK Chemie, Medical GMBH, Steinbach, Germany) and quantified. After electrophoresis on a 1% formaldehyde agarose gel, the RNA was transferred by capillary elution to a Hybond-N+membrane. Hybridization of the Northern blot was performed with full length TNF-R2 cDNA (a 1.4 kb large KpnI/HindIII fragment of pcDNAI-hTNF-R2) used as a probe. The intensity of the TNF-R2 signal, determined using a Phosphorimager and an ImageQuant® program (Molecular Dynamics, Sunnyvale, Calif.), is expressed relative to the intensity of the GAPDH signal of the same setups.

Ubiquitination Assay

2×10⁶ HEK293T cells were transiently transfected with 0.5 μg HA-ubiquitin, 0.5 μg of TNF-R2 and/or E-TRAF2 with or without 3 μg cMyc-Smurf2 WT or mutant (C716G). Twenty-four to 30 hours later, cells were lysed in TNF-R2 lysis buffer (10 mM Tris.HCl pH 7.5, 250 mM NaCl, 5 mM EDTA, 1% NP-40) supplemented with protease- and phosphatase-inhibitors. TNF-R2 or E-TRAF2 immunoprecipitation was performed with utr-1 or anti-E antibody, respectively. For endogenous TNF-R2 ubiquitination, 1×10⁷ PC60 hTNF-R55 R75 cells were used. Cells were pretreated with 30 μM MG-132 for 30 minutes prior to stimulation with 1 μg/ml hTNF for indicated times and lysed in TNF-R2 lysis buffer supplemented with protease- and phosphatase-inhibitors. Lysates were incubated with utr-4 antibody. All immunoprecipitates were immobilized on protein A trisacryl beads. Beads were washed twice with lysis buffer, twice with lysis buffer containing 0.75 M NaCl and twice with lysis buffer. Bound proteins were eluated with Laemmli buffer and analyzed by immunoblotting with indicated antibodies.

Cell Fractionation Assay

2×10⁵ HEK293T cells were transiently transfected with 100 ng TNF-R2 with or without 1 μg HA-Smurf2 WT or cMyc-Smurf2 (C716G). Forty-eight hours later, cells were lysed in 100 μl RIPA buffer (1×PBS, 1% NP-40, 0.1% SDS, 0.5% sodium deoxycholate) supplemented with protease- and phosphatase-inhibitors for 15 minutes at 4° C. The soluble fraction was separated from the insoluble fraction by centrifugation for ten minutes at 4° C. at 14,000 rpm in an Eppendorf centrifuge. 100 μl 2× Laemmli was added to the supernatant fractions, whereas cell pellets were washed with lysis buffer and resolved in 200 μl 1× Laemmli. Total cell lysates were obtained by immediate lysis of cells in 200 μl 1× Laemmli. 50 μl of each fraction was used for SDS-PAGE followed by immunoblotting with indicated antibodies.

For the cell fractionation assay of PC60hTNF-R55 R75 cells, 2×10⁶ cells were pretreated for 30 minutes with 20 μM MG-132 prior to stimulation with 1 μg/ml hTNF for indicated times. Cell lysis was performed in TNF-R2 lysis buffer supplemented with protease and phosphatase inhibitors. The assay was continued as described for HEK293T.

NF-κB Reporter Gene Assay

To determine NF-κB activation, 2×10⁵ HEK293T cells were grown in six-well plates and transiently transfected by DNA calcium phosphate coprecipitation method. The next day, 1/10^{th} of the cells were seeded out in 24-well plates in triplicate. Forty-eight hours after transfection, cells were lysed in 200 μl lysis buffer (25 mM Tris-phosphate pH 7.8, 2 mM dithiotreitol, 2 mM 1,2-cyclohexanediaminetetraacetic acid, 10% glycerol and 1% Triton X-100). Luciferase and β-galactosidase activities were analyzed as described previously (Carpentier et al., 1998). Luciferase values were normalized for β-galactosidase values to correct for differences in transfection efficiency.

p38 MAPK Activity Assay

[2×10⁵ HEK293T cells were grown in six-well plates and transiently transfected by DNA calcium phosphate coprecipitation method. Twenty-four hours later, cells were left untreated or treated with 1000 IU/ml hTNF for ten minutes, rinsed quickly with ice cold phosphate buffered saline (PBS) and lysed on ice in 200 μl SDS Sample buffer (62.5 mM Tris-HCl (pH 6.8), 2% w/v SDS, 10% glycerol, 50 mM dithiotreitol, 0.01% bromophenol blue). Cell lysates were separated on 10% SDS-PAGE and immunoblotted for detection of phosphorylated p38 MAPK or total p38 MAPK expression with phospho-p38 MAPK (Thr180/Tyr182) antibody or p38 MAPK antibody, respectively, according to manufacturer's conditions (Cell Signaling Technology, Beverly, Mass., USA).

### Example 1

### Smurf2 is a Novel TRAF2-binding Protein

TRAF2 was used as bait in a Sos recruitment system (SRS) yeast two-hybrid screening of a human spleen cDNA library to search for novel TRAF2 interaction partners. From 3.7×10⁶ screened clones, 23 positive clones expressed known interaction partners such as TRAF2 itself (6×), TRAF1 (7×), TNF-R-associated death domain protein (TRADD) (5×), cellular FLICE inhibitory protein (c-FLIP) (1×) and the TNF-R superfamily receptors CD40 (3×) and herpes virus entry mediator (HVEM) (1×). Six clones represented novel TRAF2 interacting proteins. One corresponded to a fragment of the E3 ubiquitin ligase Smurf2, which has previously been shown to play a role in TGFβ signaling (Kavsak et al., 2000; Lin et al., 2000). This clone (amino acids 385-748) contained nearly the whole catalytic HECT domain (amino acids 368-748). The latter domain contains two potential TRAF2 binding sites ([PSAT]-X-[QE]-E), a SREE motif (amino acids 398-401) and an AIEE motif (amino acids 738-741) (Ye et al., 1999).

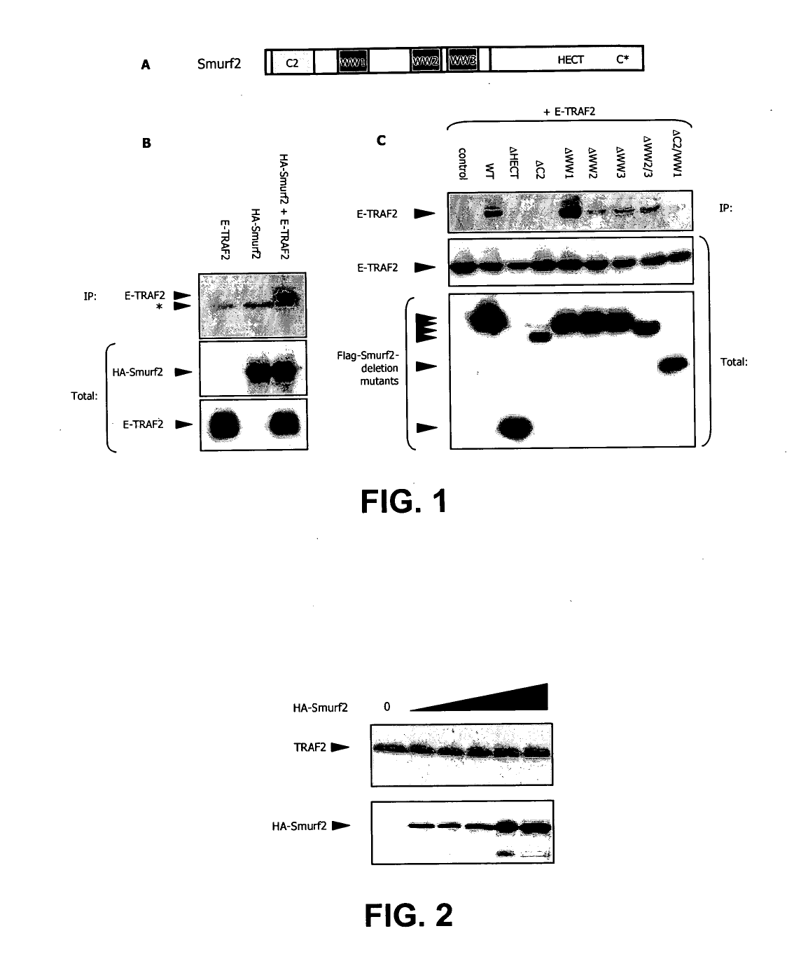

Full length Smurf2 also interacted with TRAF2 in mammalian cells, since TRAF2 was able to co-immunoprecipitate with Smurf2 in HEK293T cells that were transiently transfected with an expression plasmid for HA-tagged Smurf2 and E-tagged TRAF2 (FIG. 1, Panel B). Deletion mutants of Smurf2 lacking the C2 domain (ΔC2), the first, second or third WW domains or combinations (ΔWW1, ΔWW2, ΔWW3, ΔWW2/3), the C2 domain and the first WW domain (ΔC2/WW1) or the HECT domain (AHECT), were used to define the interaction domain(s) of Smurf2 with TRAF2 (see also, FIG. 1, Panel A). Smurf2 with a deletion of one or more WW domains (ΔWW1, ΔWW2, ΔWW3, ΔWW2/3) still retained its ability to interact with TRAF2, although deletion of WW2, WW3, or their combination, significantly reduced the binding to TRAF2. In contrast, interaction was even improved when deleting the first WW-domain. Deletion of the C2 or HECT domain abolished completely the interaction with TRAF2, indicating a requirement for the C2 domain and the HECT domain. The role of the HECT domain is consistent with the yeast two-hybrid data, in which the HECT domain was isolated as a TRAF2-interacting partner (FIG. 1, Panel C). The interaction between TRAF2 and Smurf2 could not be improved by the use of a ligase inactive Smurf2 (C716G) mutant, in which the cysteine that is believed to conjugate ubiquitin was replaced by a glycine (Huibregtse et al., 1995).

### Example 2

### Smurf2 Depletes TNF-R2 from the Cytoplasm

Smurf2 is an E3 ubiquitin ligase for Smad 1, −2, −7 and TGF-β Receptor-1 (TβR-I), leading to their proteolysis (Lin et al., 2000; Kavsak et al., 2000; Zhang et al., 2001). Likewise, the ubiquitin E3 ligases cellular inhibitor of apoptosis (cIAP) 1 and Siah1a have been implicated in the degradation of TRAF2 (Li et al., 2002; Habelhah et al., 2002). Therefore, it was assessed whether Smurf2 could also target TRAF2 for degradation. For this purpose, HEK293T cells were transiently transfected with increasing amounts of a Smurf2 expression plasmid and endogenous TRAF2 expression levels were detected by immunoblotting. Cell lysis was performed in RIPA buffer, as this leaves relatively little insoluble material. No obvious effect of Smurf2 on TRAF2 protein levels could be seen (FIG. 2). Similarly, no effect of Smurf2 could be seen on the expression of TRAF2, when the corresponding TRAF2 expression vector was cotransfected.

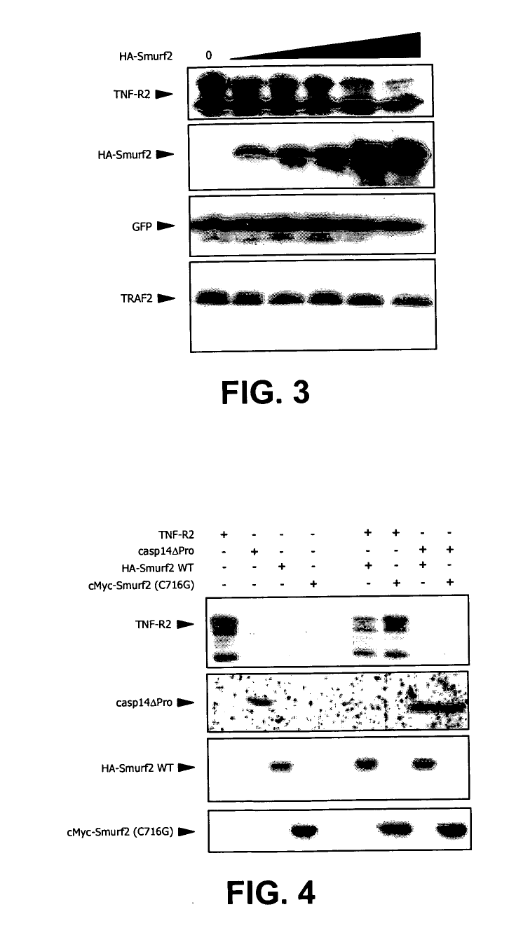

Upon receptor stimulation of CD30, CD40, TNF-R2 or latent membrane protein-1 (LMP-1), TRAF2 interacts transiently with these receptors at the plasma membrane, followed by a cytoplasmic depletion of TRAF2 and a redistribution of TRAF2 to a detergent-insoluble fraction reminiscent of membrane rafts. CD30, CD40 and TNF-R2, but not LMP-1 triggering subsequently led to TRAF2 degradation (Duckett and Thompson, 1997; Hostager et al., 2000; Chan and Lenardo, 2000; Brown et al., 2001; Fotin-Mleczek et al., 2002). In the case of CD40, TRAF2 degradation has been proposed to be mediated by the RING finger-mediated ubiquitin ligase activity of TRAF2 itself (Brown et al., 2002), whereas cIAP1 or Siah1a have been implicated in TRAF2 degradation in response to TNF-R2 or stress conditions, respectively (Li et al., 2002; Habelhah et al., 2002). In this context, it was examined whether the HECT ubiquitin ligase Smurf2 could induce the down-regulation of TRAF2 in the presence of TNF-R2. HEK293T cells were cotransfected with equal amounts of TNF-R2 and increasing amounts of Smurf2. As a control for transfection efficiency, cytoplasmic GFP was also cotransfected. Despite the presence of TNF-R2, TRAF2 degradation could not be seen when Smurf2 was cotransfected (FIG. 3). However, Smurf2 induced a dose-dependent depletion of the TNF-R2 protein. A similar Smurf2-induced decrease of the steady-state level of TPR-1 has previously been observed in the presence of Smad7 (Kavsak et al., 2000). However, in this case, no TRAF2 cotransfection was required for the Smurf2-mediated down-modulation of TNF-R2, probably because HEK293T cells already express enough endogenous TRAF2.

To analyze whether the ligase activity of Smurf2 is needed to induce the cytoplasmic down-regulation of TNF-R2, the effect of co-expression of TNF-R2 with Smurf2 wild-type (WT) or a mutant Smurf2 (C716G), which was designed to abolish the E3 ligase activity of Smurf2 (Zhang et al., 2001), on TNF-R2 expression was verified. In contrast to wild-type Smurf2, the Smurf2 (C716G) mutant did not affect the steady-state levels of TNF-R2 (FIG. 4), indicating that the TNF-R2 down-regulation is dependent on the catalytic activity of Smurf2. As a negative control, the effect of Smurf2 on the expression of another gene (caspase-14 without prodomain (casp 14Δpro)) that had been cloned after the same promotor was also verified, and found to be insensitive to Smurf2 co-expression (FIG. 4).

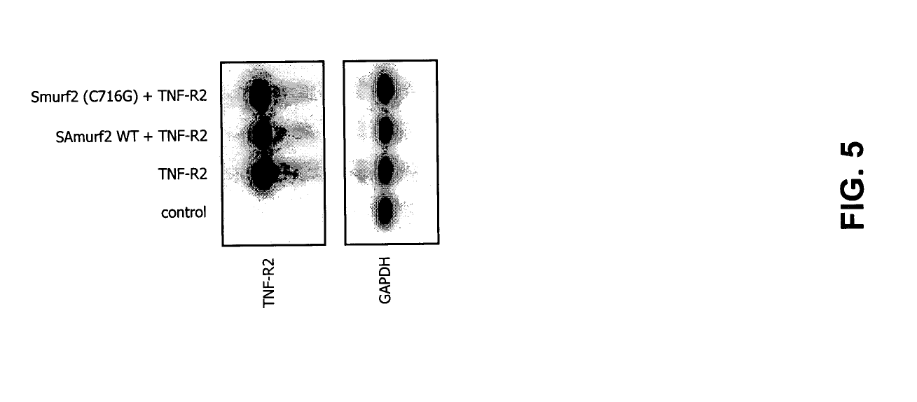

To exclude that Smurf2 might influence the steady-state level of TNF-R2 by interfering with the transcription or stability of the TNF-R2 mRNA, TNF-R2 mRNA expression was verified by Northern blotting. Therefore, RNA was isolated from HEK293T cells transiently transfected with TNF-R2, Smurf2 WT or Smurf2 (C716G), followed by Northern blot analysis with a TNF-R2-specific ³²P labeled probe. No significant difference could be noted in the mRNA levels of TNF-R2 between cells that were transfected with Smurf2 WT or Smurf2 (C716G), further indicating that Smurf2 exerts its effect on the TNF-R2 protein level (FIG. 5).

### Example 3

### TRAF2 can Function as an Adaptor Between TNF-R2 and Smurf2

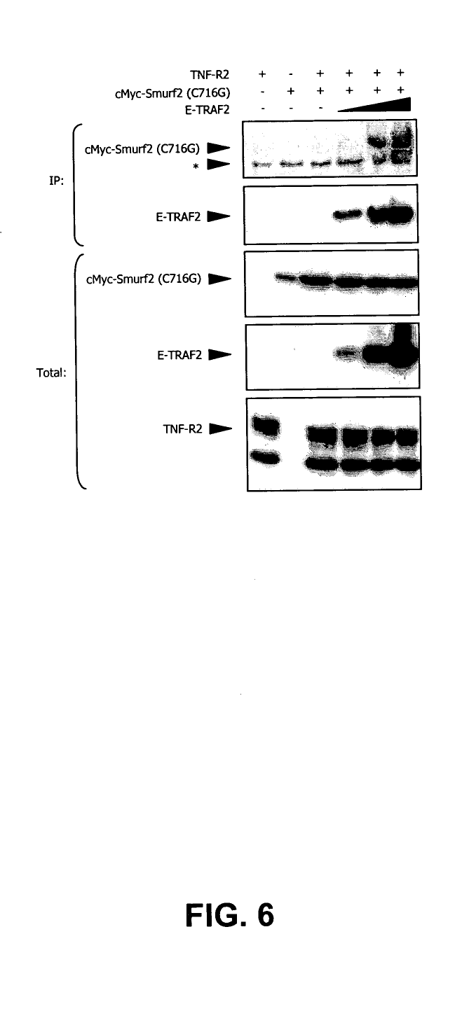

It has previously been demonstrated that Smad7 functions as an adaptor to target Smurf1 and Smurf2 to TβR-I, leading to an enhanced turnover of this receptor (Kavsak et al., 2000; Ebisawa et al., 2001). The ability of TRAF2 to bind TNF-R2 (Rothe et al., 1994) as well as Smurf2, together with the effect of Smurf2 on TNF-R2 steady-state levels, raised the interesting possibility that TRAF2 also functions to recruit Smurf2 to TNF-R2. To address this question, TNF-R2 and Smurf2 were co-expressed in HEK293T cells in the presence or absence of TRAF2, and Smurf2 co-immunoprecipitation with TNF-R2 was analyzed. To avoid Smurf2-induced TNF-R2 down-regulation, the catalytic Smurf2 (C716G) mutant was used in these experiments. When Smurf2 (C716G) was co-expressed with TNF-R2, Smurf2 (C716G) could not be co-immunoprecipitated with TNF-R2. However, in the presence of increasing amounts of TRAF2, Smurf2 co-immunoprecipitated with TNF-R2 in a dose-dependent manner, demonstrating that TRAF2 can indeed act as an adaptor between Smurf2 and TNF-R2 (FIG. 6).

### Example 4

### Smurf2 Induces Ubiquitination of TNF-R2

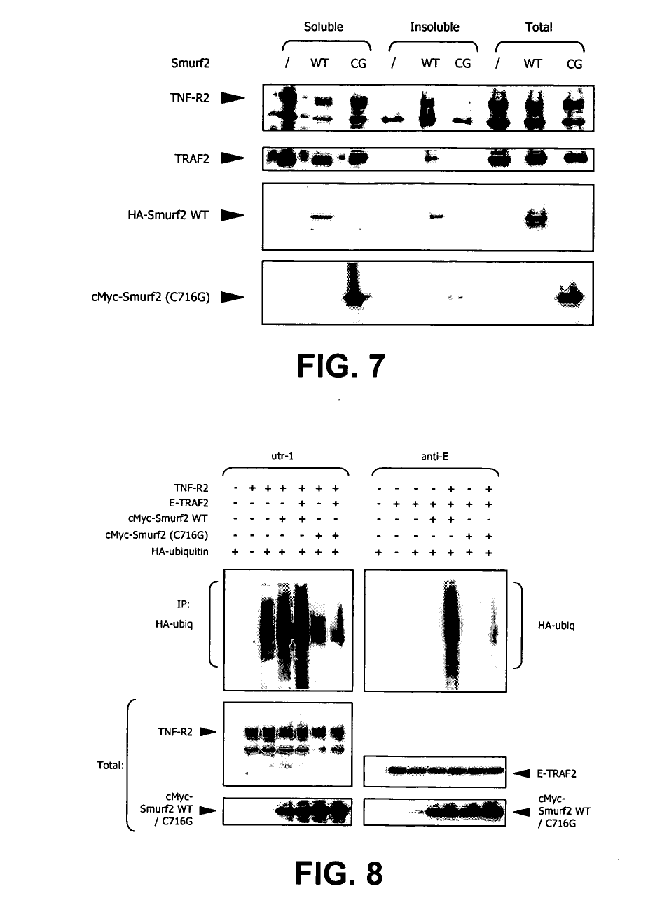

Previous data have shown that Smurf1 and Smurf2 function as E3 ligases in the ubiquitination and subsequent lysosomal and proteasomal degradation of Smad7 and TβRI (Ebisawa et al., 2001; Kavsak et al., 2000). To investigate whether TRAF2 or TNF-R2 could also undergo Smurf2-mediated ubiquitination, HEK293T cells were transiently transfected with E-TRAF2, TNF-R2 and cMyc-Smurf2 WT or mutant (C716G), together with HA-tagged ubiquitin. TNF-R2 (left panel of FIG. 7) or E-TRAF2 (right panel of FIG. 7) were immunoprecipitated, followed by Western blotting with an anti-HA antibody to detect ubiquitin-conjugates of TNF-R2 or TRAF2. In the absence of Smurf2, ubiquitination of TNF-R2 could already be observed, probably due to the presence of endogenous Smurf2 or (an)other ubiquitin ligase(s). However, TNF-R2 ubiquitination increased significantly in the presence of Smurf2 WT, which was even slightly increased upon co-expression of TRAF2. In contrast, Smurf2 (C716G) was unable to induce TNF-R2 ubiquitination and even decreased the constitutive TNF-R2 ubiquitination. The latter observation suggested that Smurf2 (C716G) can exert a dominant negative effect, possibly by competing with endogenous Smurf2. It should also be noted that a TNF-R2 mutant lacking the last 37 amino acids, disrupting the binding of TRAF2, was no longer ubiquitinated (data not shown). In contrast to TNF-R2, TRAF2 was not ubiquitinated upon Smurf2 expression (FIG. 7, right panel). Moreover, the latter experiments also showed that ubiquitinated TNF-R2 can be co-immunoprecipitated with TRAF2. In conclusion, these results further strengthen the role of Smurf2 and TRAF2 in the ubiquitination of TNFR2.

### Example 5

### Smurf2 Induces Relocalization of TNF-R2 to the Insoluble Cell Fraction

Several yeast, as well as mammalian receptors, undergo stimulus-dependent ubiquitination, which functions as a marker for internalization by endocytosis (Hicke, 2001a and b). Indeed, several mammalian receptors are able to bind directly or indirectly (“(in)directly”) to adaptin complexes that link them to clathrin-coated pits (Hicke, 1999; Strous and Govers, 1999; Sorkin and von Zastrow, 2002). On the other hand, it has been suggested that ubiquitin might affect the localization of the modified protein within the plasma membrane, e.g., relocalizing the ubiquitinated protein to membrane rafts to facilitate endocytosis. Membrane rafts are cholesterol-, sphingomyelin- and glycolipid-enriched mini-domains within the plasma membrane (Anderson and Jacobson, 2002). In this context, two interesting observations on TRAF2 and TNF-R2 have been made in the past. First, in human umbilical vein endothelial cells (HUVEC), TRAF2 has been shown to bind with the inner surface coat protein caveolin-1 of membrane invaginations. Moreover, upon overexpression of TNF-R2, TRAF2 binding to this receptor results in the recruitment of caveolin-1, possibly to localize TNF-R2 complexes to caveolae (Feng et al., 2001). In addition, it has been clearly demonstrated in several cell lines that TNF-R2 engagement leads to the cytoplasmic depletion of TRAF2, associated with its translocation to lipid rafts (Duckett and Thompson, 1997; Chan and Lenardo, 2000; Fotin-Mleczek et al., 2002). Although a main feature of lipid rafts and caveolae is their insolubility in non-ionic detergents at 4° C. (Anderson and Jacobson, 2002), TNF-R2-mediated redistribution of TRAF2 to RIPA buffer (containing sodium deoxycholate assures solubilization of rafts; Scott and Ibanez, 2001)-insoluble complexes has been observed previously, suggesting also an interaction of TRAF2 with macromolecular cell structures such as the cytoskeleton (Arch et al., 2000). Binding of TRAF2 with the actin-binding protein filamin has indeed been described (Leonardi et al., 2000). Apparently, filamin and TRAF2 are first targeted to lipid rafts by the RING finger of TRAF2, before they are sequestered into the cytoskeleton (Arron et al., 2002). Since our previous experiments were all done with the soluble cell fraction, the above findings encouraged us to assess whether the Smurf2-induced depletion of TNF-R2 did not result from its degradation, but was rather due to a TNF-R2 translocation to the insoluble fraction. To address this question, TNF-R2 expression was studied in the soluble and insoluble cell fraction prepared from HEK293T cells, which were transiently transfected with TNF-R2 in the absence or presence of Smurf2 WT or Smurf2 (C716G). Co-expression of Smurf2 WT indeed resulted in relocalization of TNF-R2 to the insoluble fraction, whereas this could not be seen upon co-expression of the ligase inactive mutant Smurf2 (C716G) (FIG. 8). Similarly, TRAF2 was also sequestered to the insoluble fraction in the presence of Smurf2 WT. Because no obvious difference in the total expression levels of TNF-R2 could be detected between the different setups, these results suggested that the down-regulation of TNF-R2 observed in the previous experiments (in which only the soluble fraction was analyzed) is due to its translocation to the insoluble fraction.

### Example 6

### TNF-R2 Undergoes a TNF-Dependent Ubiquitination and Relocalization

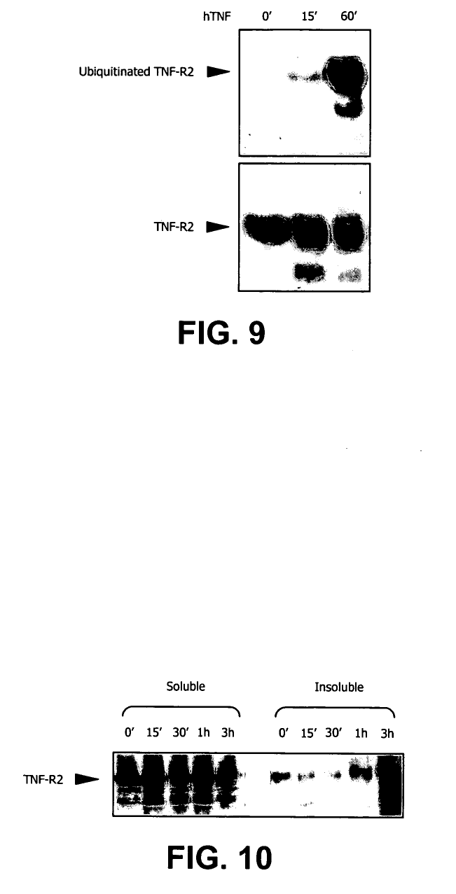

To examine whether TNF-R2 also undergoes TNF-dependent ubiquitination, ubiquitination of endogenous TNF-R2 was assessed in PC60hTNF-R55 R75 cells, a rat/T cell hybridoma cell line that contains stably transfected TNF-R1 and TNF-R2 (Vandenabeele et al., 1995). Cells were pretreated with 30 μM of the proteasome inhibitor MG-132 for 30 minutes and subsequently stimulated with 1 μg/ml hTNF for 15 or 60 minutes. TNF-R2 immunoprecipitates were analyzed for the presence of ubiquitin-TNF-R2 conjugates via immunoblotting with an anti-ubiquitin antibody. Ubiquitinated TNF-R2 could be clearly detected one hour after TNF stimulation (FIG. 9). Only one band instead of a smear was detected, thus resembling mono-ubiquitination of TNF-R2 instead of poly-ubiquitination, as has been shown for other membrane receptors (Hicke et al., 2001a). Similarly, a relocalization of TNF-R2 to the insoluble fraction could be demonstrated after three hours TNF stimulation (FIG. 10), following the time after which we were able to detect TNF-R2 ubiquitination.

### Example 7

### Smurf2 Overexpression does not affect TRAF2-or TNF-R2-induced NF-κB and p38 MAPK Activation

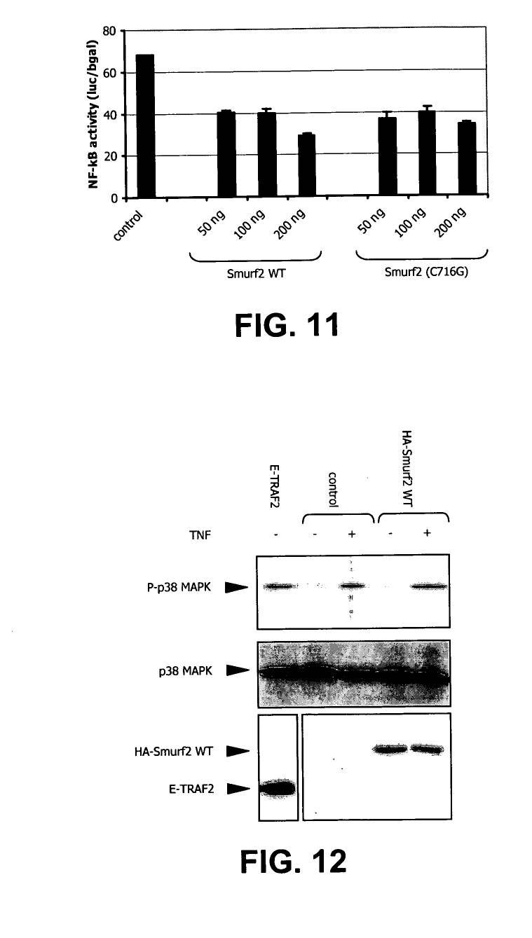

Taking into account that ectopic expression of Smurf2 can induce ubiquitination and subsequent re-localization of TNF-R2 to the insoluble fraction, we wondered whether this might have any effect on TNF-R2-mediated signal transduction leading to NF-KB and p38 MAPK activation. To investigate this, HEK293T cells were transiently transfected with TNF-R2, an NF-κB-dependent luciferase reporter gene and variable amounts of Smurf2 WT or Smurf2 (C716G) mutant. Overexpression of TNF-R2 on its own already activated NF-κB-dependent gene expression is most likely caused by the spontaneous oligomerization of receptor molecules (Rothe et al., 1995). Although Smurf2 WT induces a 40% reduction in NF-κB activity compared to the control, this inhibition seems not to be related to its ligase activity since the catalytic inactive mutant Smurf2 (C716G) gives a similar reduction (FIG. 11). It might be that Smurf2 competes with another essential signaling protein for binding to TRAF2, thus preventing NF-κB activation.

Similarly, we analyzed whether ectopic expression of Smurf2 has any effect on p38 MAPK activity in HEK293T cells. Overexpression of E-TRAF2 or TNF-stimulation for ten minutes were used as positive controls. As shown in FIG. 12, Smurf2 had no effect on basal or TNF-induced p38 MAPK phosphorylation. It should be mentioned, however, that p38 MAPK activation in the latter experiment might mainly result from TNF-R1, thus masking a potential effect of Smurf2 on TNF-R2 signaling.

### REFERENCES

### All of which are Incorporated by this Reference in their Entirety

Anderson R. G. and K. Jacobson. A role for lipid shells in targeting proteins to caveolae, rafts, and other lipid domains. Science, 2002 Jun. 7; 296(5574):1821-5.
Arch R. H., R. W. Gedrich, C. B. Thompson. Translocation of TRAF proteins regulates apoptotic threshold of cells. Biochem. Biophys. Res. Commun. 2000 Jun. 16; 272(3):936-45.
Arnett H. A., J. Mason, M. Marino, K. Suzuki, G. K. Matsushima, J. P. Ting. TNF alpha promotes proliferation of oligodendrocyte progenitors and remyelination. Nat. Neurosci. 2001, 4, 1116.
Arron J. R., Y. Pewzner-Jung, M. C. Walsh, T. Kobayashi, Y. Choi. Regulation of the subcellular localization of tumor necrosis factor receptor-associated factor (TRAF)2 by TRAF1 reveals mechanisms of TRAF2 signaling. J. Exp. Med. 2002 Oct. 7; 196(7):923-34.
Bigda J., I. Beletsky, C. Brakebusch, Y. Varfolomeev, H. Engelmann, J. Bigda, H. Holtmann, D. Wallach. Dual role of the p75 tumor necrosis factor (TNF) receptor in TNF cytotoxicity. J. Exp. Med. 1994 Aug. 1; 180(2):445-60.
Bodmer J. L., P. Schneider, J. Tschopp. The molecular architecture of the TNF superfamily. Trends Biochem. Sci. 2002 January; 27(1):19-26.
Bonni S., H. R. Wang, C. G. Causing, P. Kavsak, S. L. Stroschein, K. Luo, J. L. Wrana. TGF-beta induces assembly of a Smad2-Smurf2 ubiquitin ligase complex that targets SnoN for degradation. Nat. Cell. Biol. 2001 June; 3(6):587-95.
Brown K. D., B. S. Hostager, G. A. Bishop. Differential signaling and tumor necrosis factor receptor-associated factor (TRAF) degradation mediated by CD40 and the Epstein-Barr virus oncoprotein latent membrane protein 1 (LMPI). J. Exp. Med. 2001 Apr. 16; 193(8):943-54.
Brown K. D., B. S. Hostager, G. A. Bishop. Regulation of TRAF2 signaling by self-induced degradation. J. Biol. Chem. 2002 May 31; 277(22):19433-8.
Carpentier I., R. Beyaert. TRAF1 is a TNF inducible regulator of NF-kappaB activation. FEBS Lett. 1999 Oct. 29; 460(2):246-50.
Carpentier I., W. Declercq, N. L. Malinin, D. Wallach, W. Fiers, R. Beyaert. TRAF2 plays a dual role in NF-kappa B-dependent gene activation by mediating the TNF-induced activation of p38 MAPK and IkappaB kinase pathways. FEBS Lett. 1998 Mar. 27; 425(2):195-8.
Carpentier I., S. Janssens, K. Heyninck, R. Beyaert. NF-κB activation by tumor necrosis factor and IL-1. In: Nuclear Factor kappaB. Regulation and disease. Rudi Beyaert, Ed., Kluwer Academic Publishers, Dordrecht, The Netherlands (2003): 49-67.
Chan F. K. and M. J. Lenardo. A crucial role for p80 TNF-R2 in amplifying p60 TNF-R1 apoptosis signals in T lymphocytes. Eur. J. Immunol. 2000 February; 30(2):652-60.
Chung J. Y., Y. C. Park, H. Ye, H. Wu. All TRAFs are not created equal: common and distinct molecular mechanisms of TRAF-mediated signal transduction. J. Cell Sci. 2002 Feb. 15; 115(Pt 4):679-88.
Cope A. P., D. Aderka, M. Doherty, H. Engelmann, D. Gibbons, A. C. Jones, F. M. Brennan, R. N. Maini, D. Wallach, M. Feldmann. Increased levels of soluble tumor necrosis factor receptors in the sera and synovial fluid of patients with rheumatic diseases. Arthritis Rheum. 1992 October; 35(10):1160-9.
de Beaux A. C., A. S. Goldie, J. A. Ross, D. C. Carter, K. C. Fearon. Serum concentrations of inflammatory mediators related to organ failure in patients with acute pancreatitis. Br. J. Surg. 1996 March; 83(3):349-53.
Declercq W., G. Denecker, W. Fiers, P. Vandenabeele. Cooperation of both TNF receptors in inducing apoptosis: involvement of the TNF receptor-associated factor binding domain of the TNF receptor 75. J. Immunol. 1998 Jul. 1; 161(1):390-9.
Douni E. and G. Kollias. A critical role of the p75 tumor necrosis factor receptor (p75TNF-R) in organ inflammation independent of TNF, lymphotoxin alpha, or the p55TNF-R. J. Exp. Med. 1998, 188, 1343.
Duckett C. S. and C. B. Thompson. CD30-dependent degradation of TRAF2: implications for negative regulation of TRAF signaling and the control of cell survival. Genes Dev. 1997 Nov. 1; 11(21):2810-21.
Ebisawa T., M. Fukuchi, G. Murakami, T. Chiba, K. Tanaka, T. Imamura, K. Miyazono. Smurf1 interacts with transforming growth factor-beta type I receptor through Smad7 and induces receptor degradation. J. Biol. Chem. 2001 Apr. 20; 276(16):12477-80.
Erikstein B. K., E. B. Smeland, H. K. Blomhoff, S. Funderud, K. Prydz, W. Lesslauer, T. Espevik. Independent regulation of 55-kDa and 75-kDa tumor necrosis factor receptors during activation of human peripheral blood B lymphocytes. Eur. J. Immunol. 1991, 21, 1033.
Feng X., M. L. Gaeta, L. A. Madge, J. H. Yang, J. R. Bradley, J. S. Pober. Caveolin-1 associates with TRAF2 to form a complex that is recruited to tumor necrosis factor receptors. J. Biol. Chem. 2001 Mar. 16; 276(11):8341-9.
Fotin-Mleczek M., F. Henkler, D. Samel, M. Reichwein, A. Hausser, I. Parmryd, P. Scheurich, J. A. Schmid, H. Wajant. Apoptotic crosstalk of TNF receptors: TNF-R2-induces depletion of TRAF2 and IAP proteins and accelerates TNF-R1-dependent activation of caspase-8. J. Cell. Sci. 2002 Jul. 1; 115(Pt 13):2757-70.
Gabay C., N. Cakir, F. Moral, P. Roux-Lombard, O. Meyer, J. M. Dayer, T. Vischer, H. Yazici, P. A. Gueme. Circulating levels of tumor necrosis factor soluble receptors in systemic lupus erythematosus are significantly higher than in other rheumatic diseases and correlate with disease activity. J. Rheumatol. 1997 February; 24(2):303-8.
Gehr G., R. Gentz, M. Brockhaus, H. Loetscher, W. Lesslauer. Both tumor necrosis factor receptor types mediate proliferative signals in human mononuclear cell activation. J. Immunol. 1992, 149, 911.
Glickman M. H. and A. Ciechanover. The ubiquitin-proteasome proteolytic pathway: destruction for the sake of construction. Physiol. Rev. 2002 April; 82(2):373-428.
Grell M., P. Scheurich, A. Meager, K. Pfizenmaier. TR60 and TR80 tumor necrosis factor (TNF)-receptors can independently mediate cytolysis. Lymphokine Cytokine Res. 1993 June; 12(3):143-8.
Grosen E. A., G. A. Granger, M. Gatanaga, E. K. Ininns, C. Hwang, P. DiSaia, M. Berman, A. Manetta, D. Emma, T. Gatanaga. Measurement of the soluble membrane receptors for tumor necrosis factor and lymphotoxin in the sera of patients with gynecologic malignancy. Gynecol. Oncol. 1993 July; 50(1):68-77.
Habelhah H., I. J. Frew, A. Laine, P. W. Janes, F. Relaix, D. Sassoon, D. D. Bowtell, Z. Ronai. Stress-induced decrease in TRAF2 stability is mediated by Siah2. EMBO. J. 2002 Nov. 1; 21(21):5756-65.
Haridas V., B. G. Darnay, K. Natarajan, R. Heller, B. B. Aggarwal. Overexpression of the p80 TNF receptor leads to TNF-dependent apoptosis, nuclear factor-kappa B activation, and c-Jun kinase activation. J. Immunol. 1998 Apr. 1; 160(7):3152-62.
Heller R. A., K. Song, N. Fan, D. J. Chang. The p70 tumor necrosis factor receptor mediates cytotoxicity. Cell 1992 Jul. 10; 70(1):47-56.
Herbein G., U. Mahlknecht, F. Batliwalla, P. Gregersen, T. Pappas, J. Butler, W. A. O'Brien, E. Verdin. Apoptosis of CD8+ T cells is mediated by macrophages through interaction of HIV gp120 with chemokine receptor CXCR4. Nature 1998 Sep. 10; 395(6698):189-94.
Hicke L. Gettin' down with ubiquitin: turning off cell-surface receptors, transporters and channels. Trends Cell Biol. 1999 March; 9(3):107-12.
Hicke L. A new ticket for entry into budding vesicles-ubiquitin. Cell 2001a Sep. 7; 106(5):527-30.
Hicke L. Protein regulation by monoubiquitin. Nat. Rev. Mol. Cell Biol. 2001b March;2(3): 195-201.
Hober D., S. Benyoucef, A. S. Delannoy, D. de Groote, F. Ajana, Y. Mouton, P. Wattre. High plasma level of soluble tumor necrosis factor receptor type II (soluble TNFRII) in asymptomatic HIV-1-infected patients. Infection 1996 May-June; 24(3):213-7.
Holtmann M. H., E. Douni, M. Schutz, G. Zeller, J. Mudter, H. A. Lehr, J. Gerspach, P. Scheurich, P. R. Galle, G. Kollias, M. F. Neurath. Tumor necrosis factor-receptor 2 is up-regulated on lamina propria T cells in Crohn's disease and promotes experimental colitis in vivo. Eur. J. Immunol. 2002, 32, 3142.
Hostager B. S., I. M. Catlett, G. A. Bishop. Recruitment of CD40 and tumor necrosis factor receptor-associated factors 2 and 3 to membrane microdomains during CD40 signaling. J. Biol. Chem. 2000 May 19; 275(20):15392-8.
Huibregtse J. M., M. Scheffner, S. Beaudenon, P. M. Howley. A family of proteins structurally and functionally related to the E6-AP ubiquitin-protein ligase. Proc. Natl. Acad. Sci. U.S.A. 1995 Mar. 28; 92(7):2563-7.
Jupp O. J., S. M. McFarlane, H. M. Anderson, A. F. Littlejohn, A. A. Mohamed, R. H. MacKay, P. Vandenabeele, D. J. MacEwan. Type II tumor necrosis factor-alpha receptor (TNFR2) activates c-Jun N-terminal kinase (JNK) but not mitogen-activated protein kinase (MAPK) or p38 MAPK pathways. Biochem. J. 2001 Nov. 1; 359(Pt 3):525-35.
Kavsak P., R. K. Rasmussen, C. G. Causing, S. Bonni, H. Zhu, G. H. Thomsen, J. L. Wrana. Smad7 binds to Smurf2 to form an E3 ubiquitin ligase that targets the TGF beta receptor for degradation. Mol. Cell. 2000 December; 6(6): 1365-75.
Kim E. Y. and H. S. Teh. TNF type 2 receptor (p75) lowers the threshold of T cell activation. J. Immunol. 2001, 167, 6812.
Kollias G., E. Douni, G. Kassiotis, D. Kontoyiannis. The function of tumor necrosis factor and receptors in models of multi-organ inflammation, rheumatoid arthritis, multiple sclerosis and inflammatory bowel disease. Ann. Rheum. Dis. 1999 November; 58 Suppl 1:132-9.
Kyriakis J. M. and J. Avruch. Mammalian mitogen-activated protein kinase signal transduction pathways activated by stress and inflammation. Physiol. Rev. 2001 April; 81(2):807-69.
Leeuwenberg J. F., L. J. van Tits, T. M. Jeunhomme, W. A. Buurman. Evidence for exclusive role in signaling of tumor necrosis factor p55 receptor and a potentiating function of p75 receptor on human endothelial cells. Cytokine 1995 July; 7(5):457-62.
Leonardi A., H. Ellinger-Ziegelbauer, G. Franzoso, K. Brown, U. Siebenlist. Physical and functional interaction of filamin (actin-binding protein-280) and tumor necrosis factor receptor-associated factor 2. J. Biol. Chem. 2000 Jan. 7; 275(1):271-8.
Li X., Y. Yang, J. D. Ashwell. TNF-RII and c-IAP1 mediate ubiquitination and degradation of TRAF2. Nature 2002 Mar. 21; 416(6878):345-7.
Lin X., M. Liang, X. H. Feng. Smurf2 is a ubiquitin E3 ligase mediating proteasome-dependent degradation of Smad2 in transforming growth factor-beta signaling. J. Biol. Chem. 2000 Nov. 24; 275(47):36818-22.
Lucas R., J. Lou, D. R. Morel, B. Ricou, P. M. Suter, G. E. Grau. TNF receptors in the microvascular pathology of acute respiratory distress syndrome and cerebral malaria. J. Leukoc. Biol. 1997 May; 61(5):551-8.
Marinos G., N. V. Naoumov, S. Rossol, F. Torre, P. Y. Wong, H. Gallati, B. Portmann, R. Williams. Tumor necrosis factor receptors in patients with chronic hepatitis B virus infection. Gastroenterology, 1995 May; 108(5):1453-63.
Mason A. T., D. W. McVicar, C. A. Smith, H. A. Young, C. F. Ware, J. R. Ortaldo. Regulation of NK cells through the 80-kDa TNFR (CD120b). J. Leukoc. Biol. 1995, 58, 249.
Medvedev A. E., A. Sundan, T. Espevik. Involvement of the tumor necrosis factor receptor p75 in mediating cytotoxicity and gene regulating activities. Eur. J. Immunol. 1994 November; 24(11):2842-9.
Nophar Y., O. Kemper, C. Brakebusch, H. Englemann, R. Zwang, D. Aderka, H. Holtmann, D. Wallach. Soluble forms of tumor necrosis factor receptors (TNF-Rs). The cDNA for the type I TNF-R, cloned using amino acid sequence data of its soluble form, encodes both the cell surface and a soluble form of the receptor. EMBO J. 1990 October; 9(10):3269-78.
O'Mahoney J. V. and T. E. Adams. Optimization of experimental variables influencing reporter gene expression in hepatoma cells following calcium phosphate transfection. DNA Cell Biol. 1994 December; 13(12):1227-32.
Pickart C. M. Mechanisms underlying ubiquitination. Annu. Rev. Biochem. 2001; 70:503-33.
Reinhard C., B. Shamoon, V. Shyamala, L. T. Williams. Tumor necrosis factor alpha-induced activation of c-jun N-terminal kinase is mediated by TRAF2. EMBO J. 1997 Mar. 3; 16(5): 1080-92.
Rothe M., V. Sarma, V. M. Dixit, D. V. Goeddel. TRAF2-mediated activation of NF-kappa B by TNF receptor 2 and CD40. Science 1995 Sep. 8; 269(5229):1424-7.
Rothe M., S. C. Wong, W. J. Henzel, D. V. Goeddel. A novel family of putative signal transducers associated with the cytoplasmic domain of the 75 kDa tumor necrosis factor receptor. Cell 1994 Aug. 26; 78(4):681-92.
Schroder J., F. Stuber, H. Gallati, F. U. Schade, B. Kremer. Pattern of soluble TNF receptors I and II in sepsis. Infection 1995 May-June; 23(3):143-8.
Scott R. P. and C. F. Ibanez. Determinants of ligand binding specificity in the glial cell line-derived neurotrophic factor family receptor alpha S. J. Biol. Chem. 2001 Jan. 12; 276(2): 1450-8.
Sorkin A. and M. Von Zastrow. Signal transduction and endocytosis: close encounters of many kinds. Nat. Rev. Mol. Cell. Biol. 2002 August; 3(8):600-14.
Strous G. J. and R. Govers. The ubiquitin-proteasome system and endocytosis. J. Cell Sci. 1999 May; 112 (Pt 10):1417-23.
Tartaglia L. A., R. F. Weber, I. S. Figari, C. Reynolds, M. A. Palladino, Jr., D. V. Goeddel. The two different receptors for tumor necrosis factor mediate distinct cellular responses. Proc. Natl. Acad. Sci. U.S.A. 1991, 88, 9292.
Tartaglia L. A., D. V. Goeddel, C. Reynolds, I. S. Figari, R. F. Weber, B. M. Fendly, M. Palladino, Jr. Stimulation of human T-cell proliferation by specific activation of the 75-kDa tumor necrosis factor receptor. J. Immunol. 1993, 151, 4637.
Tsitsikov E. N., D. Laouini, I. F. Dunn, T. Y. Sannikova, L. Davidson, F. W. Alt, R. S. Geha. TRAF1 is a negative regulator of TNF signaling. Enhanced TNF signaling in TRAF1-deficient mice. Immunity 2001 October; 15(4):647-57.
Vandenabeele P., W. Declercq, B. Vanhaesebroeck, J. Grooten, W. Fiers. Both TNF receptors are required for TNF-mediated induction of apoptosis in PC60 cells. J. Immunol. 1995 Mar. 15; 154(6):2904-13.
Weiss T., M. Grell, B. Hessabi, S. Bourteele, G. Muller, P. Scheurich, H. Wajant. Enhancement of TNF receptor p60-mediated cytotoxicity by TNF receptor p80: requirement of the TNF receptor-associated factor-2 binding site. J. Immunol. 1997 Mar. 1; 158(5):2398-404.
Weiss T., M. Grell, K. Siemienski, F. Muhlenbeck, H. Durkop, K. Pfizenmaier, P. Scheurich, H. Wajant. TNFR80-dependent enhancement of TNFR60-induced cell death is mediated by TNFR-associated factor 2 and is specific for TNFR60. J. Immunol. 1998 Sep. 15; 161(6):3136-42.
Ye H., Y. C. Park, M. Kreishman, E. Kieff, H. Wu. The structural basis for the recognition of diverse receptor sequences by TRAF2. Mol. Cell. 1999 September; 4(3):321-30.
Zhang Y., C. Chang, D. J. Gehling, A. Hemmati-Brivanlou, R. Derynck. Regulation of Smad degradation and activity by Smurf2, an E3 ubiquitin ligase. Proc. Natl. Acad. Sci. U.S.A. 2001 Jan. 30; 98(3):974-9.
Zheng L., G. Fisher, R. E. Miller, J. Peschon, D. H. Lynch, M. J. Lenardo. Induction of apoptosis in mature T cells by tumor necrosis factor. Nature 1995 Sep. 28; 377(6547):348-51.
Zhu H., P. Kavsak, S. Abdollah, J. L. Wrana, G. H. Thomsen. A SMAD ubiquitin ligase targets the BMP pathway and affects embryonic pattern formation. Nature 1999 Aug. 12; 400(6745):687-93.

What is claimed is:

1. A tumor necrosis factor receptor 2 (TNF-R2) comprising SEQ ID NO:1 or a functional fragment thereof,
wherein said TNF-R2 or functional fragment is ubiquitinated.

2. The ubiquitinated TNF-R2 of claim 1, wherein said ubiquitinated TNF-β2 is isolated.

3. The ubiquitinated TNF-R2 of claim 1, wherein said TNF-R2 consists of SEQ ID NO:1.

4. The ubiquitinated TNF-R2 of claim 3, wherein said ubiquitinated TNF-R2 is isolated.

5. A process for producing ubiquinated tumor necrosis factor receptor 2 (TNF-R2), said process comprising:
ubiquinating TNF-R2 in the presence of Smurf2.

6. A method of mediating tumor necrosis factor receptor 2 (TNF-R2) ubiquitination, said method comprising:
ubiquinating TNF-R2 in the presence of tumor necrosis factor receptor-associated factor 2 (TRAF2) so as to mediate the TNF-R2 ubiquitination.

7. A method of relocalizing tumor necrosis factor receptor 2 (TNF-R2) to an insoluble cell fraction, said method comprising:
ubiquinating TNF-R2 or using Smurf2 and/or tumor necrosis factor receptor-associated factor 2 (TRAF2),
so as to relocalize TNF-R2 to the insoluble cell fraction.

8. The method according to claim 7 wherein tumor necrosis factor receptor 2 (TNF-R2) ubiquitination affects relocalization of the TNF-R2 to the insoluble cell fraction.

9. The method according to claim 7 wherein Smurf2, tumor necrosis factor receptor-associated factor 2 (TRAF2), or Smurf2 and TRAF2 affects relocalization of the TNF-R2 to the insoluble cell fraction.

10. A method of modulating tumor necrosis factor receptor 2 (TNF-R2) signaling activity, said method comprising:
ubiquinating TNF-R2 or using Smurf2 and/or tumor necrosis factor receptor-associated factor 2 (TRAF2) and/or Smurf2 or TRAF2 modulating reagents
so as to modulate the TNFR-2 signaling activity.

11. The method according to claim 10 wherein TNF-R2 ubiquitination modulates the TNFR-2 signaling activity.

12. The method according to claim 10 wherein Smurf2 and/or TRAF2 and/or Smurf2 or TRAF2 modulating reagents modulate the TNFR-2 signaling activity.

13. A method of modulating apoptosis in a cell, said method comprising:
modulating apoptosis with tumor necrosis factor receptor 2 (TNF-R2) ubiquitination so as to modulate apoptosis in the cell.

14. A method of modulating inflammation or auto-immunity associated with a disease state in a subject, said method comprising:
ubiquinating tumor necrosis factor receptor 2 (TNF-R2) in the subject so as to modulate inflammation or auto-immunity.

15. The method according to claim 14, wherein said disease state is selected from the group consisting of Crohn's disease, rheumatoid arthritis, systemic lupus erythematosus, sepsis, chronic hepatitis virus infection, acute pancreatitis, acute respiratory distress syndrome, and AIDS.

16. A method of modulating cell proliferation, said method comprising:
ubiquinating tumor necrosis factor receptor 2 (TNF-R2) in a cell population
so as to modulate cell proliferation.
